(19) United States
(12) Patent Application Publication
(10) Pub. No.: US 2018/0250328 A1
(43) Pub. Date: Sep. 6, 2018

(54) Metallocene-boronic acid-containing compounds and copolymers as antimicrobial agents.

(71) Applicant: University of South Carolina, Columbia, SC (US)

(72) Inventors: Chuanbing Tang, Columbia, SC (US); Peng Yang, Columbia, SC (US)

(21) Appl. No.: 15/932,256

(22) Filed: Feb. 16, 2018

Related U.S. Application Data
(60) Provisional application No. 62/467,438, filed on Mar. 6, 2017.

(51) Int. Cl. A61K 31/80; A61P 31/04; C08F 230/04; A61K 31/43

(52) U.S. Cl. CPC A61K 31/80; A61K 31/43; C08F 230/04; A61P 31/04

(57) ABSTRACT

Polymeric compounds for targeting broad-spectrum bacterial strains are provided. The polymeric compounds can include at least one metallocene monomeric unit and at least one boronic acid monomeric unit. The metallocene monomeric unit can include a cationic metallocene moiety paired to an anion, and the anion can be an anionic antibiotic compound.

### CROSS-REFERENCE TO A RELATED APPLICATION

The present application claims priority to U.S. Provisional Patent Application Ser. No. 62/467,438 titled “METALLOCENE-BORONIC ACID-CONTAINING COMPOUNDS AND COPOLYMERS AS ANTIMICROBIAL AGENTS” of Tang, et al. filed on Mar. 6, 2017, the disclosure of which is incorporated by reference herein.

### GOVERNMENT SUPPORT CLAUSE

This invention was made with government support under R01A1120987 awarded by National Institutes of Health. The government has certain rights in the invention.

### FIELD OF INVENTION

This present disclosure relates to copolymers having antibacterial properties. More specifically, the present disclosure relates to metallocene-boronic acid-containing compounds and copolymers as antimicrobial agents.

### BACKGROUND

Bacterial infections have become one of the most urgent global health threats, leading to increased healthcare costs, destruction of local tissues, patient disability, morbidity, and even death. If no effective strategies are taken to prevent and treat bacterial infections, it is estimated that by 2050 they could claim 10 million lives and cost up to 100 trillion dollars globally. However, commonly used antibiotics, such as penicillin and methicillin, have shown diminished antimicrobial efficacy, and numerous bacterial pathogens have accumulated multidrug resistance (MDR).

Multidrug-resistant Gram-negative bacteria are posing an increasingly alarming threat, making many last-resort antibiotics ineffective. Compared to therapies for Gram-positive strains, a recent analysis showed very few antibiotics in development could be promising for fighting these life-threatening Gram-negative bacterial infections. Due to having double cell membranes as an intrinsic defense, it is difficult for antibiotics to not only inhibit critical bacterial processes, but also penetrate two membrane barriers and escape efflux pumps. In many cases, these agents can cross the outer membrane, but stop short of penetrating the cytoplasmic membrane. Meanwhile, the antimicrobial agent must overcome efflux pumps even after the penetration of two membranes. With the frequency of MDR increasing at an alarming rate, there is an urgent need to develop new antimicrobial agents. It would be most desirable to have multiple pathogen-specific therapeutics that can target various types of bacteria, but especially Gram-negative bacteria. As such, the development of compounds that are effective against broad-spectrum bacterial strains including those with multidrug resistance would be useful.

### BRIEF SUMMARY

Aspects and advantages of embodiments of the present disclosure will be set forth in part in the following description, or may be learned from the description, or may be learned through practice of the embodiments.

Polymeric compounds for targeting broad-spectrum bacterial strains are provided. The polymeric compounds can include at least one metallocene monomeric unit and at least one boronic acid monomeric unit. The metallocene monomeric unit can include a cationic metallocene moiety paired to an anion, and the anion can be an anionic antibiotic compound. The metallocene-boronic acid-containing copolymers can enhance interactions with bacterial cells and therefore improve antimicrobial effectiveness of antibiotics against not only Gram-positive bacterial strains but also Gram-negative bacterial strains. The cationic metallocene moiety can be attracted to negatively-charged bacterial membranes via electrostatic interaction. The boronic acid group can bind with peptidoglycan and polysaccharides on bacterial cell walls/membranes through the formation of reversible boronic esters. Thus, bacteria can be rapidly captured and cell membranes disrupted while the antibiotic targets and kills the bacteria. The metallocene-boronic acid-containing compounds and polymers demonstrate synergistic antimicrobial effects and excellent bactericidal function with broad spectrum activity against various strains of bacteria. Further, the compounds exhibit minimal toxicity and non-hemolytic activity in vitro and in vivo on mammalian cells.

### BRIEF DESCRIPTION OF THE DRAWINGS

A full and enabling disclosure of the present invention, including the best mode thereof to one skilled in the art, is set forth more particularly in the remainder of the specification, which includes reference to the accompanying figures.

FIG. 1A illustrates an exemplary anion-paired metallocene-containing compound, where M is a metal, X is an anion, and R₁ is a substituted group (e.g., an organic group).

FIG. 1B illustrates an exemplary anion-paired metallocene-containing monomer, where M is a metal and X is an anion, and R₂ is a substituted group (e.g., an organic group).

FIG. 2A illustrates an exemplary boronic acid-containing compound, where R₃ is a substituted group (e.g., an organic group).

FIG. 2B illustrates an exemplary boronic acid-containing-containing monomer, where R₄ is a substituted group (e.g., an organic group).

FIG. 3 shows an exemplary metallocene-boronic acid-containing copolymer, where M is a metal and X is an anion, and R₂ and R₄ are substituted groups (e.g., organic groups).

FIG. 4 shows an antibiotic-loaded metallocene-containing copolymer, with the antibiotic shown as penicillin or other beta-lactam related compounds containing anionic groups, where M is a metal and R₂, R₄, and R₅ represent functional groups (e.g., organic functional groups).

FIG. 5 shows the antimicrobial effects of a cobaltocenium-boronic acid-containing copolymer (PCo-PPB-1, about 20 wt. % boronic acid monomer; PCo-PPB-2, about 15 wt. % boronic acid monomer; and PCo-PPB-3, about 8 wt. % boronic acid monomer), a cobaltocenium-containing homopolymer (PCo), and a boronic acid-containing homopolymer (PPB) against Gram-positive bacteria S. aureus and Gram-negative bacteria E. coli using disk-diffusion assays.

FIG. 6A shows minimum inhibitory concentration (MIC) evaluation of several cobaltocenium-boronic acid-containing copolymers (PCo-PPB-1, about 20 wt. % boronic acid monomer; PCo-PPB-2, about 15 wt. % boronic acid monomer; and PCo-PPB-3, about 8 wt. % boronic acid monomer) and a metallocene-containing homopolymer (PCo) against S. aureus.

FIG. 6B shows minimum inhibitory concentration (MIC) evaluation of several cobaltocenium-boronic acid-containing copolymers (PCo-PPB-1, about 20 wt. % boronic acid monomer; PCo-PPB-2, about 15 wt. % boronic acid monomer; and PCo-PPB-3, about 8 wt. % boronic acid monomer) and a boronic acid-containing homopolymer (PCo) against E. coli.

FIG. 7A shows Fourier-transform infrared spectroscopy (FTIR) spectra of peptidoglycan, cobaltocenium-boronic acid-containing copolymers (PCo-PPB), and cobaltocenium-boronic acid-containing copolymer-peptidoglycan conjugates (PCo-PPB-peptidoglycan).

FIG. 7B shows Fourier-transform infrared spectroscopy (FTIR) spectra of lipopolysaccharide, cobaltocenium-boronic acid-containing copolymers (PCo-PPB), and cobaltocenium-boronic acid-containing copolymer lipopolysaccharide conjugates (PCo-PPB-lipopolysaccharide).

FIG. 8A shows an agar diffusion test of the antimicrobial activity of penicillin, penicillin loaded cobaltocenium-containing polymers (PCo-Peni), and penicillin loaded cobaltocenium-boronic acid-containing copolymers (PCo-PPB-Peni) against six strains of bacteria using different amounts of penicillin (2, 5, and 10 m).

FIG. 8B is a 3-D plot of inhibition zones of penicillin, penicillin loaded cobaltocenium-containing polymers (PCo-Peni), and penicillin loaded cobaltocenium-boronic acid-containing copolymers (PCo-PPB-Peni) against six strains of bacteria using different amounts of penicillin (A: 5 μg penicillin-G alone, B: PCo-Peni with 5 μg penicillin-G, C: PCo-PPB-Peni with 5 μg penicillin-G, D: 10 μg penicillin-G, E: PCo-Peni with 10 μg penicillin-G, F: PCo-PPB-Peni with 10 μg penicillin-G).

FIG. 9 shows confocal laser scanning microscopy (CLSM) images of a control, cobaltocenium-boronic acid-containing copolymers (PCo-PPB, 11 μg/mL), penicillin (5 μg/mL), and penicillin loaded cobaltocenium-boronic acid-containing-copolymers (PCo-PPB-Peni, 16 μg/mL, with the concentration of penicillin at 5 μg/mL) against six strains of bacteria (using BacLight live/dead stain, green indicates live cells, red indicates dead cells).

FIG. 10 shows field-emission scanning electron microscope (FESEM) images of control and penicillin loaded cobaltocenium-boronic acid-containing-copolymers (PCo-PPB-Peni, 16 μg/mL, with the concentration of penicillin-G at 5 μg/mL) against six strains of bacteria (all scale bars are 2.0 μm).

FIG. 11 illustrates the antimicrobial mechanism of cobaltocenium-boronic acid-containing copolymers with antibiotic bioconjugates against different types of bacteria.

FIG. 12A shows ultraviolet-visible spectroscopy (UV-vis) absorption of nitrocefin solution with different amounts of cobaltocenium-boronic acid-containing copolymers (100, 200, and 400 μg PCo-PPB) after adding β-lactamase for 1 h.

FIG. 12B shows an image of nitrocefin solutions with different amounts of cobaltocenium-boronic acid-containing copolymers (measured in micrograms) after adding β-lactamase for 1 h.

FIG. 13 is a graph illustrating apoptosis detection after staining with Annexin V for phosphate-buffered saline (PBS) and cobaltocenium-boronic acid-containing copolymers PCo-PPB-1 (about 20 wt. % boronic acid monomer), PCo-PPB-2 (about 15 wt. % boronic acid monomer), and PCo-PPB-3 (about 8 wt. % boronic acid monomer).

FIG. 14 shows average percentages of splenocytes and apoptotic cells after injecting with phosphate-buffered saline (PBS) or the cobaltocenium-boronic acid-containing-copolymers (PCo-PPB-1, 2 and 3) at a concentration of 10 mg/kg body weight in vivo.

FIG. 15 shows the average percentages of different cell types (i.e., T cell populations (CD3+, CD4+ and CD8+) or B cells (CD19+)) after staining. Mice were injected with either with phosphate-buffered saline (PBS) or the cobaltocenium-boronic acid-containing-copolymers (PCo-PPB-1, 2 and 3) at a concentration of 10 mg/kg body weight and then analyzed by flow cytometry for the different types of lymphocyte populations in the spleen after 48 h.

FIG. 16 shows hemolysis percentages of cobaltocenium-boronic acid-containing copolymers (PCo-PPB-1, 2 and 3) at varying concentrations (10, 50, 100 and 500 μg) as well as 0.5% Triton-X100 as a control.

### DETAILED DESCRIPTION

Reference now will be made to the embodiments of the invention, one or more examples of which are set forth below. Each example is provided by way of an explanation of the invention, not as a limitation of the invention. In fact, it will be apparent to those skilled in the art that various modifications and variations can be made in the invention without departing from the scope or spirit of the invention. For instance, features illustrated or described as one embodiment can be used on another embodiment to yield still a further embodiment. Thus, it is intended that the present invention cover such modifications and variations as come within the scope of the appended claims and their equivalents. It is to be understood by one of ordinary skill in the art that the present discussion is a description of exemplary embodiments only, and is not intended as limiting the broader aspects of the present invention, which broader aspects are embodied exemplary constructions.

Chemical elements are discussed in the present disclosure using their common chemical abbreviation, such as that commonly found on a periodic table of elements. For example, hydrogen is represented by its common chemical abbreviation H, helium is represented by its common chemical abbreviation IIe, and so forth.

As used herein, the term “polymer” generally includes, but is not limited to, homopolymers; copolymers, such as, for example, block, graft, random and alternating copolymers; and terpolymers; and blends and modifications thereof. Furthermore, unless otherwise specifically limited, the term “polymer” shall include all possible geometrical configurations of the material. These configurations include, but are not limited to isotactic, syndiotactic, and random symmetries.

The term “organic” is used herein to refer to a class of chemical compounds that are comprised of carbon atoms. For example, an “organic polymer” is a polymer that includes carbon atoms in the polymer backbone, but may also include other atoms either in the polymer backbone and/or in side chains extending from the polymer backbone (e.g., oxygen, nitrogen, sulfur, etc.).

The term “pharmaceutically effective amount” refers to an amount of a drug or pharmaceutical agent that will elicit the biological or medical response of a tissue, system, animal, or human that is being sought by a researcher or clinician. This amount can be a therapeutically effective amount.

Embodiments of the present disclosure include metallocene-boronic acid-containing copolymers. The metallocene-boronic acid-containing copolymers can be used in biomedical applications such as drugs and antimicrobial agents. The metallocene-boronic acid-containing copolymers can act as effective antimicrobial agents against a broad spectrum of bacterial pathogens, including Gram-positive bacteria, Gram-negative bacteria, and bacteria that has shown resistance to conventional antibiotics.

A metallocene is a compound having two cyclopentadienyl anions (Cp, which is C₅H₅⁻) bound to a metal center (M) in the oxidation state II, with the resulting general formula (C₅H₅)₂M. Closely related to the metallocenes are the metallocene derivatives, (e.g. titanocene dichloride, vanadocene dichloride). However, a metallocene-containing cationic compound generally has a positive charge due to the metal center (M) being in the oxidation state I. Thus, the overall charge of the metallocene-containing cationic compound is +1, such that the metallocene-containing cationic compound is paired to an anion having a negative charge, such as hexafluorophosphate (PF₆⁻), tetraphenylborate (BPh₄⁻), tetrafluoroborate (BFᵣ⁻), trifluoromethanesulfonate (OTf), F⁻, Cl⁻, Br⁻, I⁻, NO₃⁻, acetate (Ac⁻), sulfate (SO₄²⁻), hydrogen sulfate (HSO₄⁻), perchlorate (ClO₄⁻), bromate (BrO₃⁻), cyanide (CN⁻), thiocyanate (SCN⁻), hydroxide (OH⁻), dihydrogen phosphate (H₂PO₄⁻), or formate (HCOO⁻).

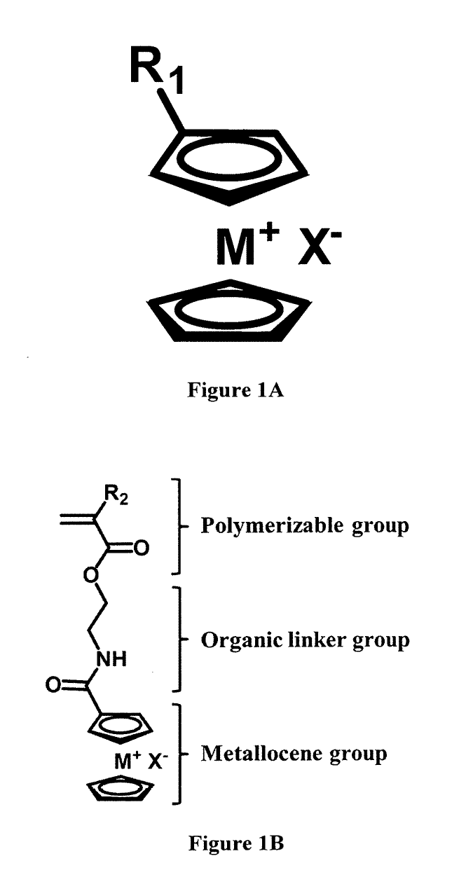

Referring to FIG. 1A, a generic anion-paired metallocene-containing compound is shown, where M is a metal, X is an anion, and R₁ is a substituted group (e.g., an organic group).

I. Anion-Paired Metallocene-Containing Monomers

Generally, each anion-paired metallocene-containing monomer includes an anion-paired metallocene group covalently attached to a polymerizable group via an organic linker group (U.S. Pat. No. 9,402,394 of Tang, et al. teaches metallocene-containing compounds, the disclosure of which is incorporated by reference herein). Referring to FIG. 1B, for example, an anion-paired metallocene-containing monomer is shown, where M is a metal and X is an anion. The anion-paired metallocene-containing monomer includes a polymerizable group covalently attached to an anion-paired metallocene group via an organic linker group.

A cationic metallocene group includes two cyclopentadienyl rings bound to a metal center (M) in an oxidation state that leaves the cationic metallocene group with a positive charge (such as +1 or +2). Thus, the cationic metallocene group is generally paired with a counter ion. For example, an anion (X) can be present such that the charge of the resulting anion-paired cationic metallocene group is zero. The metals can include, for example, iron (Fe), cobalt (Co), rhodium (Rh), ruthenium (Ru), and mixtures thereof.

The anion (“X”) paired with the cationic metallocene-containing compound can be any suitable anion, including, but not limited to, hexafluorophosphate (PF₆⁻), tetraphenylborate (BPh₄⁻), tetrafluoroborate (BF₄⁻), trifluoromethanesulfonate (OTf), F⁻, Cl⁻, Br⁻, I⁻, NO₃⁻, acetate (Ac⁻), sulfate (SO₄²⁻), hydrogen sulfate (HSO₄⁻), perchlorate (ClO₄⁻), bromate (BrO₃⁻), cyanide (CN⁻), thiocyanate (SCN⁻), hydroxide (OH⁻), dihydrogen phosphate (H₂PO₄⁻), or formate (HCOO⁻).

Various organic linker groups can be positioned between the polymerizable group (e.g., containing a vinyl group) and the anion-paired metallocene group. In one embodiment, the organic linker group includes a simple alkyl chain having a number (m) of repeating carbon atoms (e.g., —CH₂—), with m being an integer of from 1 to about 50, such as 2 to about 40, and such as from 3 to about 20. In one particular embodiment, m is from 2 to about 12. As shown in the embodiment of FIG. 1B, the organic linker group includes an ethyl chain (i.e., an alkyl chain of 2 carbons).

The organic linker group can also include any covalent linkage to one of the cyclopentadienyl rings of the anion-paired metallocene group, such as an amide linkage as shown in FIG. 1B. Although not shown in the exemplary embodiment of FIG. 1B, the alkyl chain of the organic linker group can be substituted with common substituents found on alkyl chains (e.g., hydroxyl groups, ester groups, etc.).

The polymerizable group of the anion-paired metallocene-containing monomer can include a vinyl group, such as an acrylic group, a methacrylic group, a styrenic group, an acrylamide group, or a norbornene group, etc. For example, FIG. 1B shows an exemplary monomer having a (meth)acrylic group forming its polymerizable group, with R₂ being either H (i.e., an acrylic group) or —CH₃ (i.e., a methacrylic group). No matter the particular chemistry of the polymerizable group, a vinyl group can present and configured for polymerization into a polymeric chain. FIG. 1B shows an exemplary anion-paired metallocene-containing monomer that is, when M is cobalt (Co), R₁ is a methyl group, 2-cobaltocenium amidoethyl methacrylate.

II. Boronic Acid-Containing Monomers

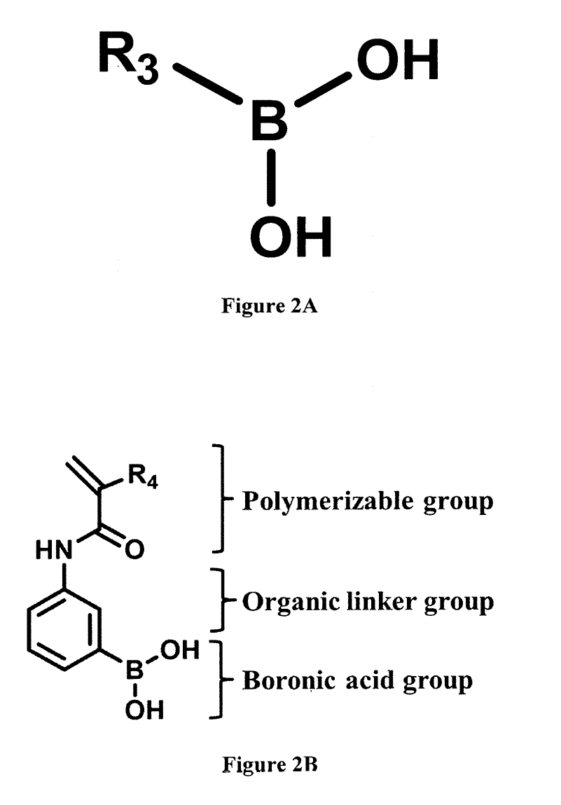

The metallocene-containing monomers can be copolymerized with boronic acid containing monomers. The boronic acids are trivalent boron-containing organic compounds that possess one alkyl substituent (i.e., a C-B bond) and two hydroxyl groups (i.e. two B-OH bonds) to fill the remaining valences on the boron atom. Referring to FIG. 2A, a generic boronic-containing compound is shown, where R₃ is a substituted group (e.g., an organic group). Generally, each boronic acid-containing monomer includes a boronic acid group covalently attached to a polymerizable group via an organic linker group. Referring to FIG. 2B, for example, a boronic acid-containing monomer is shown, where R₄ is a substituted group (e.g., an organic group).

Diverse organic linker groups can be positioned between the polymerizable group (i.e., containing the vinyl group) and the boronic acid group. In the embodiment of FIG. 1B, the organic linker group includes a phenyl ring. Although not shown in the exemplary embodiment of FIG. 1B, the organic linker group can also include any covalent linkage to the boronic acid group, such as alkyl chain, amide group, thienyl groups, ester group, etc. The alkyl chain of the organic linker group can be substituted with common substituents found on chains (e.g., hydroxyl groups, amine groups, etc.).

The polymerizable group of the boronic acid-containing monomer can include a vinyl group, such as an acrylamide group, methacrylamide group, acrylic group, a methacrylic group, a styrenic group, an acrylamide group, or a norbornene group. For example, FIG. 2B shows an exemplary monomer having a (meth)acrylamide group forming its polymerizable group, with R₄ being either H (i.e., an acrylamide group) or —CH₃ (i.e., a methacrylamide group). No matter the particular chemistry of the polymerizable group, a vinyl group can be present and configured for polymerization into a polymeric chain.

FIG. 2B shows an exemplary boronic acid-containing monomer. And, for example, when the R₄ group of FIG. 2B is an H-group, the boronic acid-containing monomer is 3-acrylamidophenylboronic acid.

III. Metallocene-Boronic Acid-Containing Compounds and Polymers

The metallocene-containing monomers and boronic acid-containing monomers can be polymerized to form metallocene-boronic acid-containing copolymers (including block copolymers, random copolymers, graft copolymers, star copolymers and/or organic/inorganic hybrids) that contain at least one unit derived from metallocene moiety and one unit derived from boronic acid moiety (i.e., at least one metallocene monomer and at least one boronic acid monomer). In one embodiment, a metallocene-boronic acid-containing copolymer can be prepared by free radical and controlled/living radical copolymerization of a vinyl-boronic acid-containing monomer and a vinyl-metallocene-containing monomer. The copolymers can have anion-paired metallocene moieties arid boronic acid moieties on the side-chains.

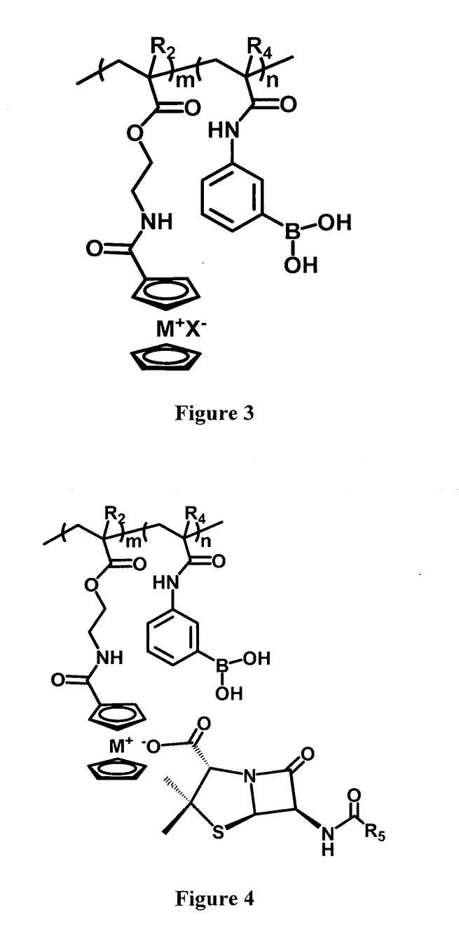

For example, referring to FIG. 3, the metallocene-containing monomer of FIG. 1B and the boronic acid-containing monomer of FIG. 2B are used as comonomers and are polymerized into a random copolymer, where m is the number of monomeric units of the metallocene-containing monomer and n is the number of monomeric units of the boronic acid-containing monomer within the copolymer. For example, n and m can each range from about 5 to about 1000, such as from about 10 to about 500, and such as about 20 to about 100. The copolymer molecular weight can have an average range of from about 1,000 g/mol to about 1,000,000 g/mol. More specifically, the copolymer molecular weight can have an average range of from about 2,000 g/mol to about 30,000 g/mol, such as from about 5,000 g/mol to about 25,000 g/mol, and such as from about 5,000 g/mol to about 25,000 g/mol.

The properties of the metallocene-boronic acid-containing copolymers can be tuned by changing the comonomer structures (the polymerizable vinyl moiety, the linker, metallocene-containing moiety or boronic acid-containing moiety), the molecular weight of the polymer, and/or the relative amounts of any comonomers present. For example, the amount of metallocene monomer can range from about 50 wt. % to about 95 wt. %, such as from about 60 wt. % to about 80 wt. %, and such as from about 65 wt. % to about 75 wt. %. Further, the amount of boronic acid monomer can range from about 5 wt. % to about 50 wt. %, such as from about 15 wt. % to about 40 wt. %, and such as from about 25 wt. % to about 35 wt. %.

The molar ratio of metallocene monomer to boronic acid monomer (mols metallocene monomer/mols boronic acid monomer) can range from about 20 to about 1, such as from about 5 to about 15, and such as from about 8 to about 12. The average number of monomers in the copolymer chain can range from about 5 to about 300, such as from about 10 to about 200, and such as from about 20 to about 100. Additionally, in some embodiments the average molecular weight of metallocene-boronic acid copolymers can range from about 2,000 g/mol to about 100,000 g/mol, such as from about 10,000 g/mol to about 75,000 g/mol, and such as from about 20,000 g/mol to about 50,000 g/mol.

IV. Metallocene-Boronic Acid-Containing Copolymers as Antimicrobial Agents

Metallocene-boronic acid-containing compounds and polymers of the present disclosure can be used as antimicrobial agents. For example, cobaltocenium-boronic acid-containing copolymers of the present disclosure have shown antimicrobial activity against a broad spectrum of bacteria, including Gram positive bacteria (S. aureus and E. faecalis), Gram negative bacteria (E. coli, K. pneumoniae, P. vulgaris, and P. aeruginosa) and drug resistant bacteria (methicillin-resistant Staphylococcus aureus, MRSA).

Such copolymers can be administered in a pharmaceutically effective amount as an antibiotic to a subject (e.g., a living subject such as a human or animal) infected with such bacteria (i.e., a metallocene-boronic acid-containing antibiotic). The metallocene-boronic acid-containing antibiotic may be administered to the subject via any suitable routes of administration, such as oral, rectal, transmucosal, transnasal, intestinal, or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Pharmaceutical compositions that include the metallocene-boronic acid-containing antibiotic may be manufactured by processes well known in the art, e.g., by means of conventional mixing, dissolving, granulating, dragee-making, levigating, emulsifying, encapsulating, entrapping or lyophilizing processes. Thus, such pharmaceutical compositions comprising the metallocene-boronic acid-containing antibiotic may be formulated in conventional manner using one or more physiologically acceptable carriers comprising excipients and auxiliaries, which facilitate processing of the active ingredients into preparations which can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, artificial cerebrospinal fluid (CSF) or physiological salt buffer. For transmucosal administration, penetrants appropriate to the barrier to be permeated can used in the formulation. Such penetrants are generally known in the art. For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol. Cellulose preparations can also be utilized such as, for example, maize starch, wheat starch, rice starch, potato starch, gelatin, gum tragacanth, methyl cellulose, hydroxypropylmethyl-cellulose, sodium carbomethylcellulose, and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Determination of a therapeutically effective amount is well within the capability of those skilled in the art, especially in light of the detailed disclosure provided herein. Toxicity and therapeutic efficacy of the metallocene-boronic acid-containing antibiotic described herein can be determined by standard pharmaceutical procedures in vitro, in cell cultures or experimental animals. The data obtained from these in vitro and cell culture assays and animal studies can be used in formulating a range of dosage for use in humans. The dosage and formulation may vary depending upon the dosage form employed and the route of administration utilized.

Depending on the severity and responsiveness of the condition to be treated, dosing can be of a single or a plurality of administrations, with the course of treatment lasting from several days to several weeks or until a cure is effected or diminution of the infected state is achieved. The amount of a metallocene-boronic acid containing antibiotic to be administered will, of course, be dependent on factors including the subject being treated, the severity of the affliction, and the manner of administration.

V. Antibiotic-Loaded Metallocene-Boronic Acid Copolymers

In one embodiment, the metallocene-boronic acid copolymers can be used to promote the effects of traditional antibiotics against a broad spectrum of bacterial pathogens including Gram positive bacteria (S. aureus and E. faecalis) and Gram negative bacteria (E. coli, K. pneumoniae, P. vulgaris, and P. aeruginosa). Metallocene-boronic acid-containing copolymers have at least one metallocene unit and one boronic acid unit (i.e., at least one metallocene monomer and at least one boronic acid monomer). The metallocene unit can have a positive charge. In such cationic metallocene-boronic acid-containing copolymers, the anion (X) as shown in FIG. 3 can be replaced with an anionic antibiotic compound. Thus, the cationic metallocene-containing moiety can be paired with an anionic antibiotic compound.

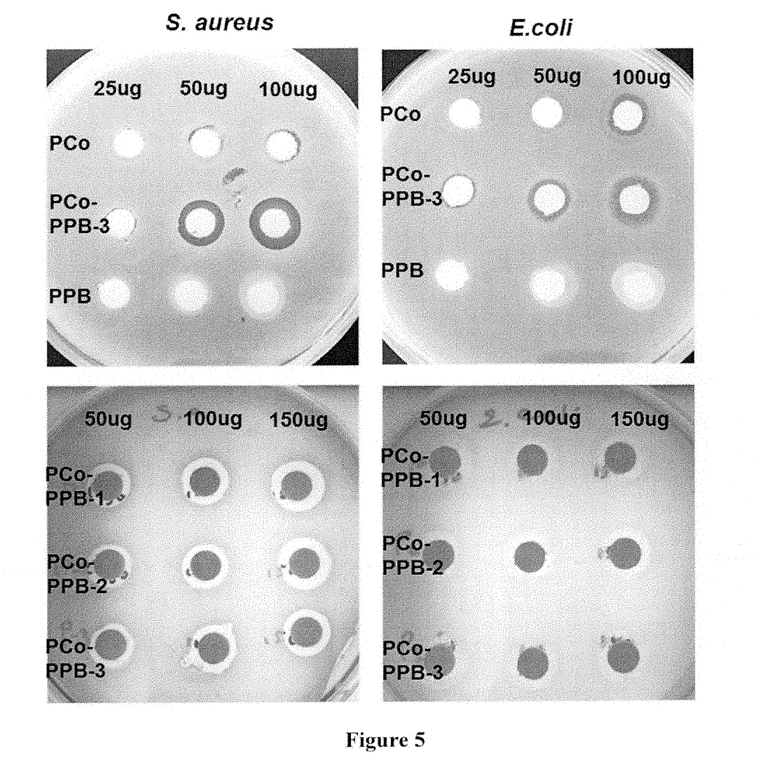

All β-lactam type antibiotics, such as penicillins, carbapenems, and cephalosporins (including the first, second, third, fourth and fifth generation), can be loaded in the metallocene-boronic acid-containing copolymers. These antibiotic-loaded metallocene-boronic acid-containing copolymers can produce excellent effects against Gram-negative and Gram-positive bacteria, and especially drug resistant bacteria. For example, FIG. 4 shows an antibiotic-loaded metallocene-containing copolymer, with the antibiotic shown as a penicillin or a related compound, where M is a metal and R₂ R₄, and R₅ represent organic functional groups.

Thus, traditional antibiotics can be loaded in metallocene-boronic acid-containing materials and can improve antimicrobial ability against a broad spectrum of drug resistant bacterial pathogens. Exemplary antibiotics that can be paired with the cationic metallocene moiety in the metallocene-boronic acid-containing copolymer include, but are not limited to, penicillins (Penams): Amoxicillin, Ampicillin (Pivampicillin, Hetacillin, Bacampicillin, Metampicillin, Talampicillin), Epicillin, Carbenicillin (Carindacillin), Ticarcillin, Temocillin, Azlocillin, Piperacillin, Mezlocillin, Mecillinam Sulbenicillin, Clometocillin, Benzathine, benzylpenicillin, Procaine benzylpenicillin, Azidocillin, Penamecillin, Phenoxymethylpenicillin (V), Propicillin, Benzathine phenoxymethylpenicillin and Pheneticillin; cephalosporins, including the first, second, third, fourth, and fifth generations; carbapenems, including Biapenem, Ertapenem, Doripenem, Imipenem, Meropenem and Panipenem.

The properties of the antibiotic-loaded metallocene-boronic acid copolymers can be tuned by changing the comonomer structures (the polymerizable vinyl moiety, the linker, metallocene-containing moiety or boronic acid-containing moiety), the molecular weight of the polymer, the relative amounts of any comonomers, and/or the amount and type of antibiotic. For example, the antibiotic can be present in the antibiotic-loaded metallocene-boronic acid copolymer in an amount of from about 10 wt. % to about 50 wt. %, such from about 20 wt. % to about 40 wt. %, and such as from about 25 wt. % to about 35 wt. %. Further, the molar ratio of antibiotic to metallocene monomer (mols antibiotic / mols metallocene monomer) can range from about 0.30 to about 1.0, such as from about 0.45 to about 0.85, and such as from about 0.55 to about 0.75.

The amount of metallocene monomer can range from about 50 wt. % to about 95 wt. %, such as from about 60 wt. % to about 80 wt. %, and such as from about 65 wt. % to about 75 wt. %. Further, the amount of boronic acid monomer can range from about 5 wt. % to about 50 wt. %, such as from about 15 wt. % to about 40 wt. %, and such as from about 25 wt. % to about 35 wt. %.

The molar ratio of metallocene monomer to boronic acid monomer (mols metallocene monomer/mols boronic acid monomer) can range from about 20 to about 1, such as from about 5 to about 15, and such as from about 8 to about 12. The average number of monomers in the copolymer chain can range from about 5 to about 300, such as from about 10 to about 200, and such as from about 20 to about 100. Additionally, in some embodiments, the average molecular weight of antibiotic-loaded metallocene-boronic acid copolymers can range from about 2,000 g/mol to about 100,000 g/mol, such as from about 10,000 g/mol to about 75,000 g/mol, and such as from about 20,000 g/mol to about 50,000 g/mol.

The antimicrobial efficacy of various metallocene-boronic acid-containing copolymers including random copolymers, block copolymers, graft copolymers, star copolymers, and organic/inorganic hybrids was demonstrated was demonstrated through various experiments which are discussed in the Examples, below.

### EXAMPLE 1

This example demonstrates the antimicrobial efficacy of metallocene-boronic acid-containing copolymers against Gram-positive S. aureus and Gram-negative E. coli.

The cobaltocenium-boronic acid-containing copolymers (PCo-PPB) were synthesized via reversible-addition fragmentation chain transfer (RAFT) polymerization using cobaltocenium-containing monomer (2-cobaltocenium amidoethyl methacrylate) and boronic acid-containing monomer (3-acrylamidophenylboronic acid) as co-monomers. Three cobaltocenium-boronic acid-containing copolymers with different weight fractions of boronic acid were synthesized by changing molar ratios of comonomers, while keeping their molecular weight similar (Mn 14,500 g/mol). The proportion of boronic acid in the copolymers was about 20 wt. % (PCo-PPB-1), 15 wt. % (PCo-PPB-2), and 8 wt. % (PCo-PPB-3), respectively.

For the bacteria, a single colony was inoculated in 30 mL tryptic soy broth (TSB) at 37° C. for 24 hours, shaking at 190 rpm/min. All bacteria were grown to an optical density of about 1.00 (OD₆₀₀=1.00) for further use. To conduct the agar disk-diffusion assays, actively-growing cultures of each bacterial strain on mannitol salt agar (MSA) were inoculated on tryptic soy broth (TSB) agar plates. The bacterial growth culture (cell concentrations were 1.0×10⁶ CFU/mL) was diluted from 10 μL to 1 mL in tryptic soy broth (TSB) and 100 μL was spread on TSB agar plates to form a bacterial lawn covering the plate surface. Then, 6 mm (diameter) filter discs were added to the plate surface, aqueous cobaltocenium-boronic acid-containing copolymers (PCo-PPB) at different concentrations were added to disks, and the plates were incubated at 28° C. for 18 h. The development of a clear zone around the disk was indicative of the ability of the compounds to kill bacteria.

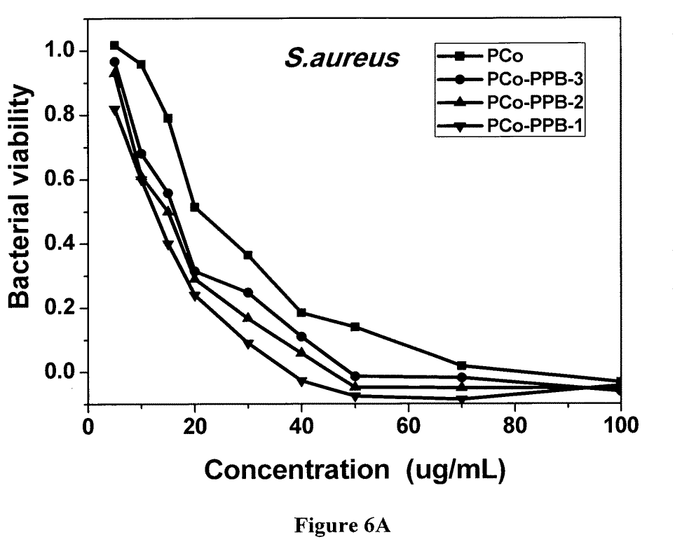

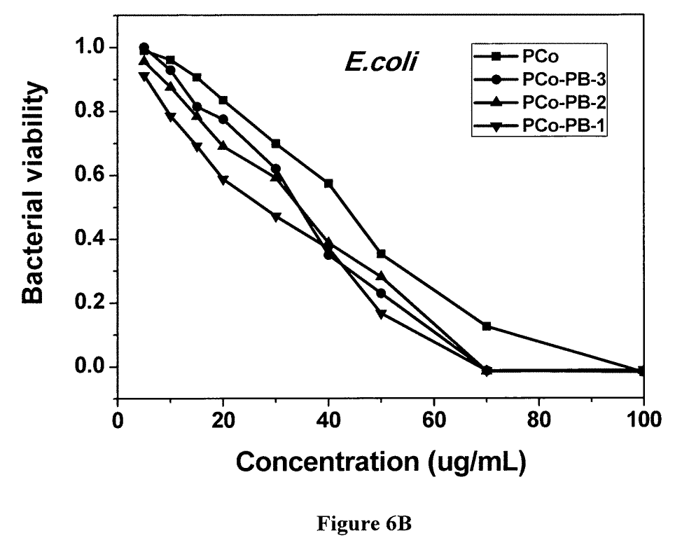

As shown in FIG. 5, compared with cobaltocenium-containing homopolymers (PCo, Mn=15,000 g/mol) and boronic acid-containing homopolymers (PPB), the cobaltocenium-boronic acid-containing copolymers (PCo-PPB) showed significantly enhanced activity against both types of bacteria. However, it was found that the activity against S. aureus was significantly higher. The minimum inhibitory concentration (MIC) of cobaltocenium-boronic acid-containing copolymers and cobaltocenium-containing homopolymer was further evaluated against the two bacteria. Fifty (50) μL aqueous solution of PCo-PPB copolymers or PCo homopolymers with different concentrations were added to 96 well plates. Then, 150 μL bacterial TSB solution (OD₆₀₀=1.00) was added to the wells. An unadulterated bacterial TSB solution was used as a control. The assay plate was incubated at 37° C. for 12 hours. Bacterial growth was detected at OD₆₀₀ and was compared to controls of bacterial TSB solution without polymers. As shown in FIGS. 6A and 6B, the cobaltocenium-boronic acid-containing copolymers exhibited higher antibacterial activity against S. aureus and E. coli than the PCo homopolymer alone. The MICs of cobaltocenium-boronic acid-containing copolymers decreased to 40-70 μg/mL, in comparison with over 100 μg/mL for cobaltocenium containing homopolymer. For the three cobaltocenium-boronic acid-containing copolymers tested, the antimicrobial efficacy increased with an increase in boronic acid content.

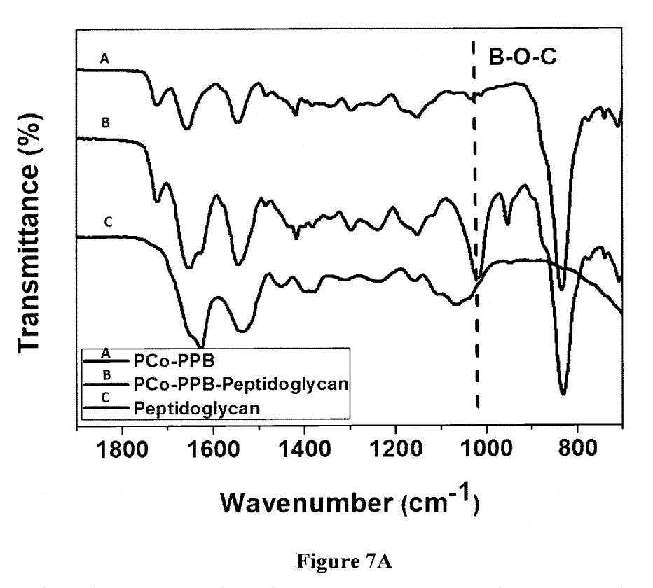

In order to verify the interaction between metallocene-boronic acid-containing copolymer and peptidoglycan as well as lipopolysaccharide, peptidoglycan, and lipopolysaccharide, extractions from cell membranes of S. aureus and E.coli were selected to model macromolecules. Firstly, the cobaltocenium-boronic acid-containing copolymer (PCo-PPB-1) and peptidoglycan (weight ratio 3:1) were mixed in dimethyl sulfoxide/water (DMSO/H₂O) solvent for 6 h at room temperature, and then employed for Fourier-transform infrared spectroscopy (FTIR) analysis after freeze-drying. Compared with the spectra of cobaltocenium-boronic acid-containing copolymer and peptidoglycan alone, the characteristic peak of boronate ester (B-O-C stretching vibration) at 1050 cm⁻¹ appeared in the spectrum of cobaltocenium-boronic acid-containing copolymer-peptidoglycan conjugates (FIG. 7A), which suggests that peptidoglycan successfully bonded with copolymers via the formation of boronate esters between boronic acids from copolymers and diols from peptidoglycan. Similarly, the peak of boronate ester was also found in the spectra of cobaltocenium-boronic acid-containing copolymer-lipopolysaccharide conjugates (FIG. 7B).

### EXAMPLE 2

The utilization of metallocene-boronic acid-containing compounds, random copolymers, block copolymers, graft copolymers, star copolymers, and organic/inorganic hybrids as drug delivery materials for traditional antibiotics was demonstrated. Different commercially available antibiotics (including all β-lactam type antibiotics, such as penicillins, carbapenems and cephalosporins, including the first, second, third, fourth, and fifth generations) were loaded with cationic metallocene-containing polymers.

The ability of cationic metallocene-containing compounds and polymers to activate conventional antibiotics against drug-resistant bacterial pathogens was demonstrated. For example, cobaltocenium-boronic acid-containing copolymers loaded with penicillin G showed antimicrobial activity against a broad spectrum of bacteria, including Gram-positive bacteria (S. aureus and E. faecalis) and Gram-negative bacteria (E. coli, P. vulgaris, P. aeruginosa and K. pneumoniae).

Penicillin-G was loaded into cobaltocenium-boronic acid-containing-copolymers to form bioconjugates (labeled as PCo-PPB-Peni) via ionic complexation between cationic cobaltocenium and anionic antibiotic. High antibiotic loading capacity (31 wt %, the molar ratio of cobaltocenium moiety to penicillin is 1:0.6) was easily obtained due to the strong electrostatic interactions.

Disk-diffusion assays were used to evaluate the antimicrobial activity of penicillin loaded-cobaltocenium-boronic acid-containing copolymers against six strains of bacteria including Gram-positive bacteria (S. aureus and E. faecalis) and Gram-negative bacteria (E. coli, P. vulgaris, P. aeruginosa and K. pneumoniae). To compare bactericidal efficiency, a penicillin loaded-cobaltocenium-containing homopolymer (named as PCo-Peni) was prepared as a control.

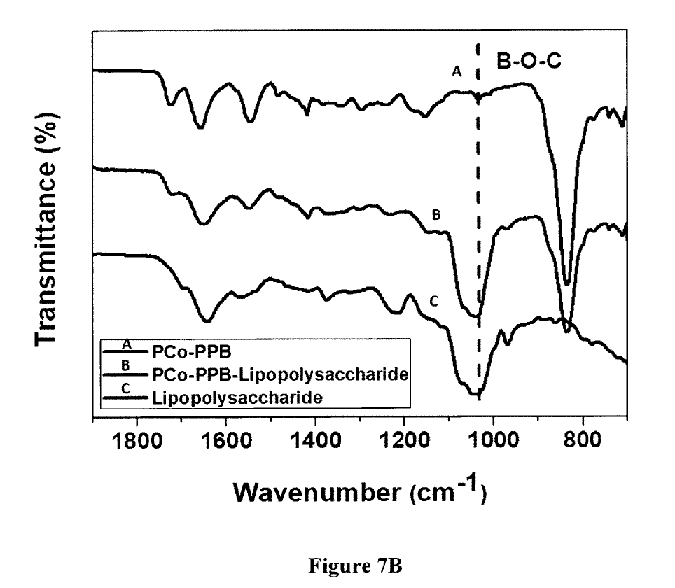

As shown in FIGS. 8A and 8B, penicillin-G (5 μg) alone showed very low antimicrobial efficacy against S. aureus, and the inhibition zone was only 7 mm. In contrast to penicillin-G, penicillin loaded-cobaltocenium-containing homopolymers and penicillin loaded-cobaltocenium-boronic acid-containing copolymers displayed distinct enhancement and the inhibition zone increased to 12 mm and 16 mm, respectively, with the same amount of penicillin-G (5 μg). By maintaining the amount of penicillin-G at 10 μg, the inhibition zone of penicillin-G, penicillin loaded-cobaltocenium-containing homopolymers, and penicillin loaded-cobaltocenium-boronic acid-containing copolymers appreciably increased to 14.5 mm, 17 mm, and 23 mm, respectively. When tested against the other five types of bacteria, penicillin loaded-cobaltocenium-boronic acid-containing copolymers exhibited the best antimicrobial results at varying amounts of penicillin.

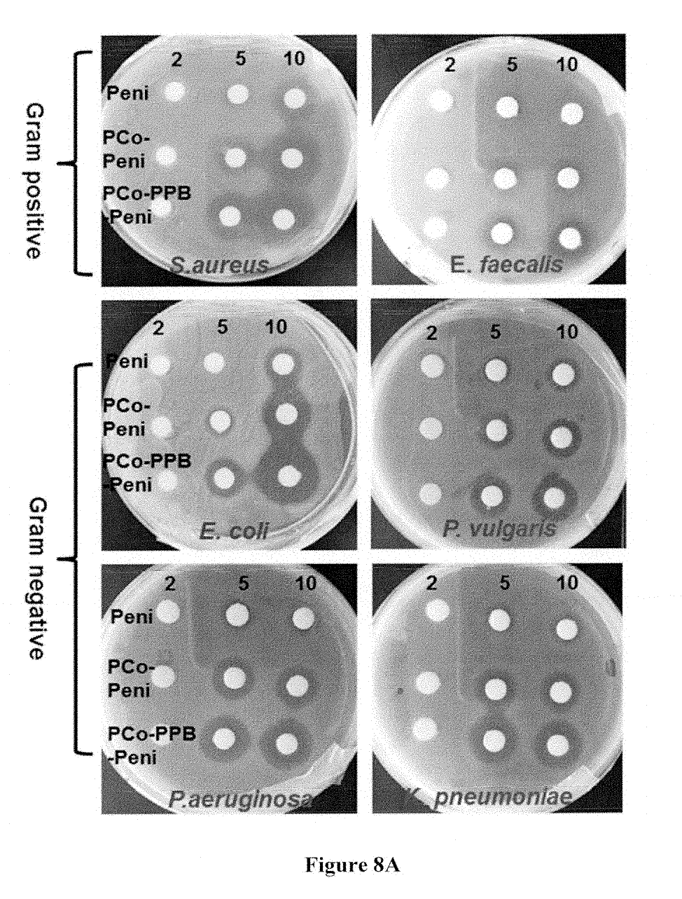

The inhibition effect of penicillin-loaded cobaltocenium-boronic acid-containing copolymers against six types of bacteria was further investigated by confocal scanning laser microscopy (CSLM). One (1) mL of active bacterial stock of various strains was introduced to 5 penicillin-G, 11 μg cobaltocenium-boronic acid-containing copolymer, and 16 μg penicillin-loaded cobaltocenium-boronic acid-containing copolymers (penicillin-G weight: 5 m), respectively. An untreated cell suspension was used as the control. Following 18-hour incubation at 37° C., 1 μL LIVE/DEAD BacLight (Bacterial Viability Kit; INVITROGEN INC.®) was added to the incubation solution. After incubation for 15 minutes, cells were imaged using a LEICA TCS SP5® Laser Scanning Confocal Microscope with a 63X oil immersion lens. When excited at 488 nm with Argon and Helium/Neon lasers, bacteria with intact membranes displayed green fluorescence (Emission=500 nm) and bacteria with disrupted membranes fluoresced red (Emission=635 nm). LIVE/DEAD bacteria viability assay by CSLM suggested penicillin-G and cobaltocenium-boronic acid-containing copolymers alone were not effective at killing bacteria at relatively low concentrations (FIG. 9). In contrast, almost all bacteria incubated with penicillin-loaded cobaltocenium-boronic acid-containing-Peni bioconjugates were killed, with CSLM displaying obvious red or yellow fluorescence from dead bacteria.

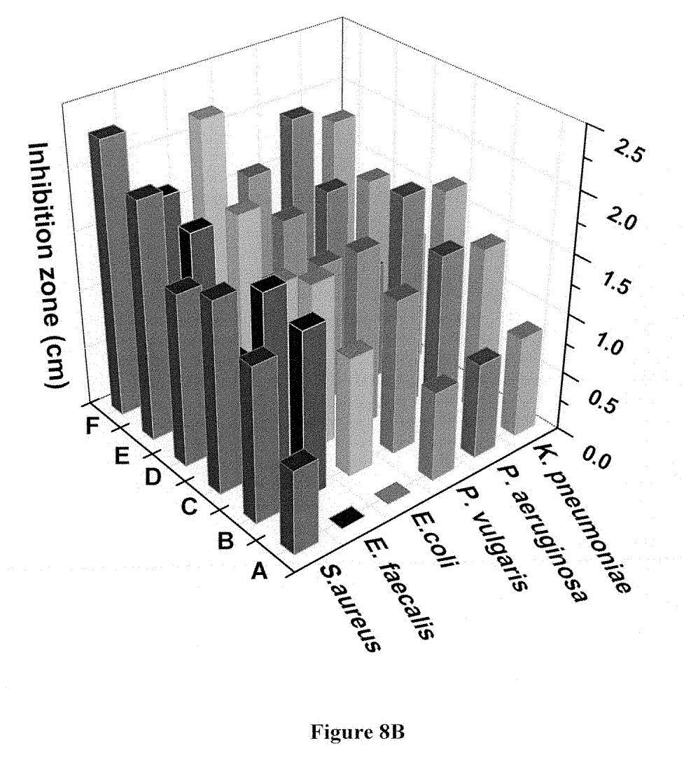

The morphologies of different bacteria after incubation with penicillin-loaded cobaltocenium-boronic acid-containing copolymers were examined by field-emission scanning electron microscopy (FESEM). Ten (10) μL of bacteria cell solution were grown overnight on one glass slide in a 12-well plate containing 1 mL of TSB medium at 37° C. Cell suspensions were diluted to OD₆₀₀=1.0. Penicillin loaded cobaltocenium-boronic acid-containing copolymer (PCo-PPB-Peni) bioconjugates (16 μg, with penicillin-G weight 5 μg) were added to the 1 mL cell stock solution and incubated at 37° C. overnight. An unadulterated cell suspension was used as a control. The samples were then fixed in cacodylate buffered with 2.5% glutaraldehyde solution (pH=7.2) for 2-3 h at 4° C. and post-fixed with 1% osmium tetraoxide at 4° C. for 1 h. The samples were dried under their critical point, then coated with gold using DENTON DESK II SPUTTER COATER® for 120 s and observed by FESEM. An untreated cell suspension was used as the control. From the FESEM images in FIG. 10, it was observed that the penicillin-loaded cobaltocenium-boronic acid-containing copolymers could damage the bacterial membranes, shrink the bacteria, and effectively kill the bacteria. In contrast, the untreated bacteria (control groups) exhibited a typical sphere or rod morphology with a smooth surface.

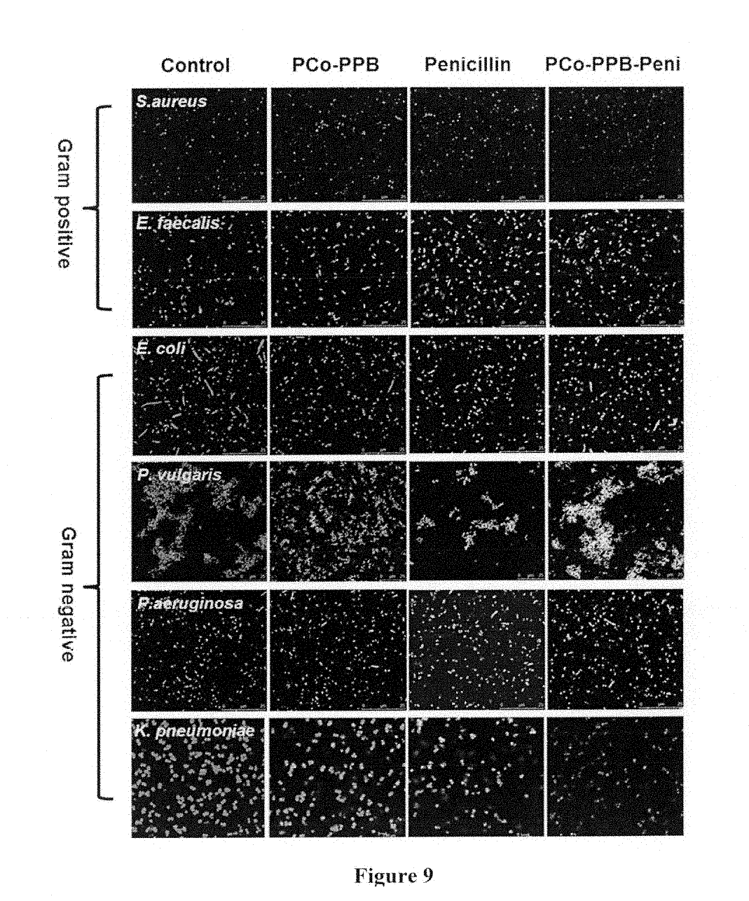

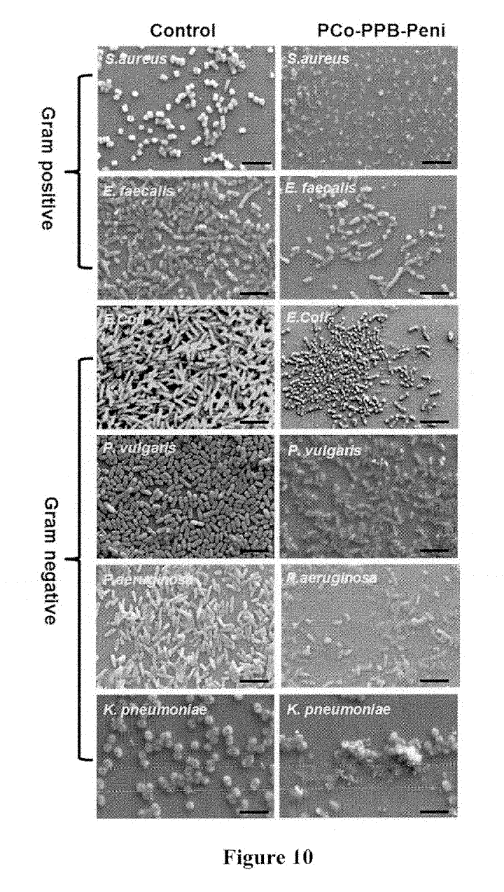

The strong bactericidal efficacy of penicillin-loaded cobaltocenium-boronic acid-containing copolymers was believed to be attributed to the synergistic effects originating from the building blocks of cobaltocenium and phenylboronic acid. FIG. 11 illustrates the antimicrobial mechanisms of penicillin-loaded cobaltocenium-boronic acid-containing copolymers against Gram-positive and Gram-negative strains of bacteria. In one aspect, the phenylboronic acid group can attach to the bacterial surface by binding with peptidoglycan or lipopolysaccharides on the surface of cells to help polymers capture various bacteria. In a second aspect, the cationic cobaltocenium not only interacts with the negatively charged bacterial membrane, but blocks the electrostatic chelation between the β-lactam antibiotic and cationic amino acid residue (such as Lys₂₃₄) of β-lactamase to keep penicillin from being hydrolyzed by bacteria enzymes. The effect of cobaltocenium-boronic acid-containing copolymers on the β-lactamase activity was investigated by UV-visible spectra using nitrocefin as an indicator (FIGS. 12A and 12B). After adding β-lactamase, the nitrocefin solution quickly turned red from yellow and an absorption peak appeared near 480 nm due to the hydrolysis of its β-lactam ring. However, when cobaltocenium-boronic acid-containing copolymers first bound with nitrocefin for the formation of conjugates, the addition of β-lactamase only caused the solution to change color very slowly with very low absorption at 480 nm. Even with the concentration of copolymers increased to 400 μg/mL, the color of the solution maintained a yellow appearance, suggesting cobaltocenium-boronic acid-containing copolymers can inhibit β-lactamase activity and prevent hydrolysis of the antibiotic's β-lactam ring.

### EXAMPLE 3

Metallocene-boronic acid-containing copolymers demonstrated high efficacy in lysing bacterial cells as well as reducing β-lactamase activity. Furthermore, the cobaltocenium-boronic acid-containing copolymers possessed excellent biocompatibility, exhibiting non-hemolytic activity and minimal in vitro and in vivo toxicity.

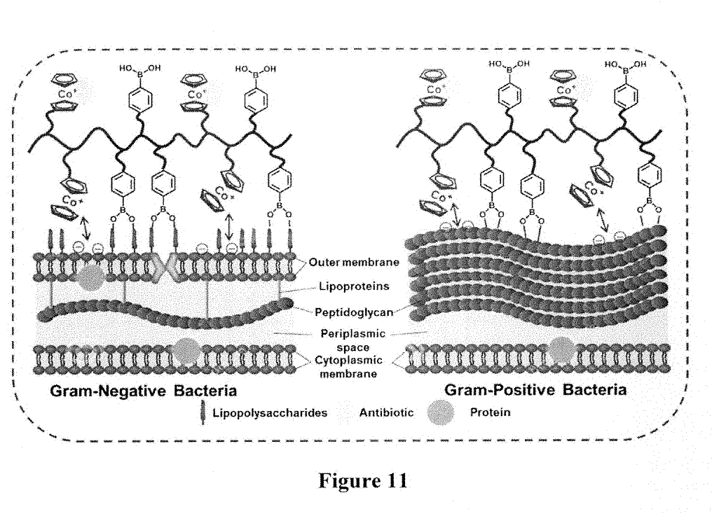

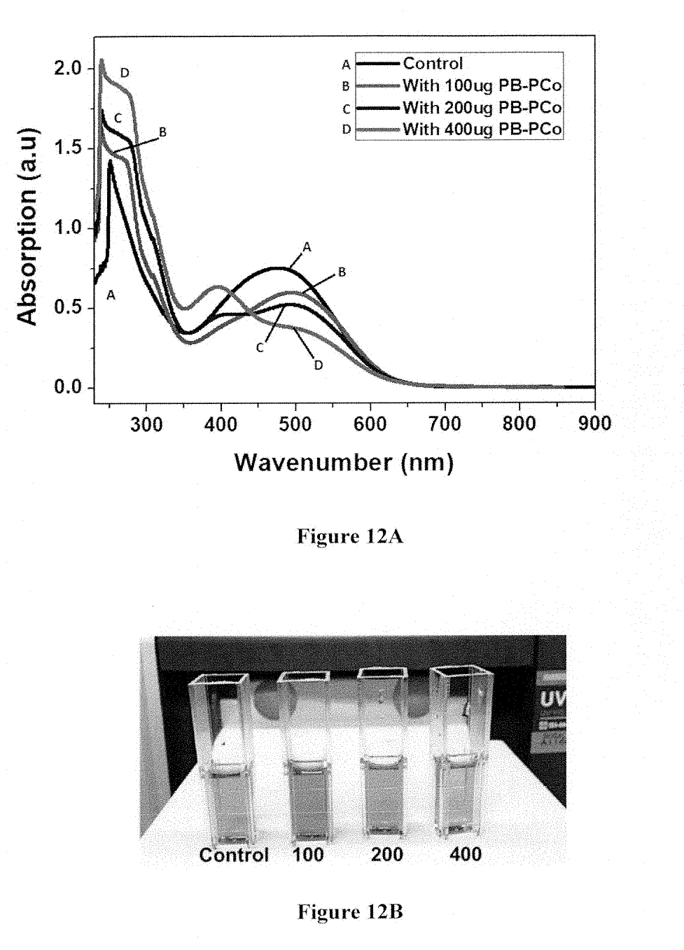

To determine the toxicity of cobaltocenium-boronic acid-containing copolymers (PCo-PPB-1, about 20 wt. % boronic acid; PCo-PPB-2, about 15 wt. % boronic acid; and PCo-PPB-3, about 8 wt. % boronic acid), both in vitro and in vivo experiments were performed to determine their ability to induce programmed cell death (known as apoptosis) in immune cells. For this purpose, the cells were cultured with phosphate-buffered saline (PBS) solution and 10 and 50 μg/mL of cobaltocenium-boronic acid-containing copolymers for 24 h, and then fluorochrome-labeled with Annexin V (a member of the annexin family of intracellular proteins that can bind to phosphatidylserine in a calcium-dependent manner), which was employed to specifically target and identify apoptotic cells. It was found that the percentages of apoptotic cells after treatment with cobaltocenium-boronic acid-containing copolymers were very similar to that of the PBS control (FIG. 13). However, it was difficult to detect apoptosis in vivo because of the clearing of apoptotic cells by phagocytic macrophages. To overcome this problem, mice were first in vivo treated with the cobaltocenium-boronic acid-containing copolymers, then apoptotic cells were cultured in vitro for 24 h and apoptosis was detected. It was found that the cobaltocenium-boronic acid-containing copolymer-treated splenocytes were susceptible to induction of apoptosis to the same extent as PBS-treated cells (FIG. 14). If the cobaltocenium-boronic acid-containing copolymers were toxic to the cells, a higher percentage of the apoptosis-positive cells would have been observed. Naive immune cells in a tissue culture medium underwent apoptosis in a fraction of the cells as shown by positivity for apoptosis in the negative controls and the PBS-treated groups. Thus, during flow cytometry analysis, all other treatment groups were gated based on the PBS-treated groups. From the in vivo studies, it was observed that the percentages of the apoptotic splenocytes from copolymer-injected and PBS-injected mice were comparable, indicating PCo-PPB copolymers presented very low cytotoxicity to immune cells.

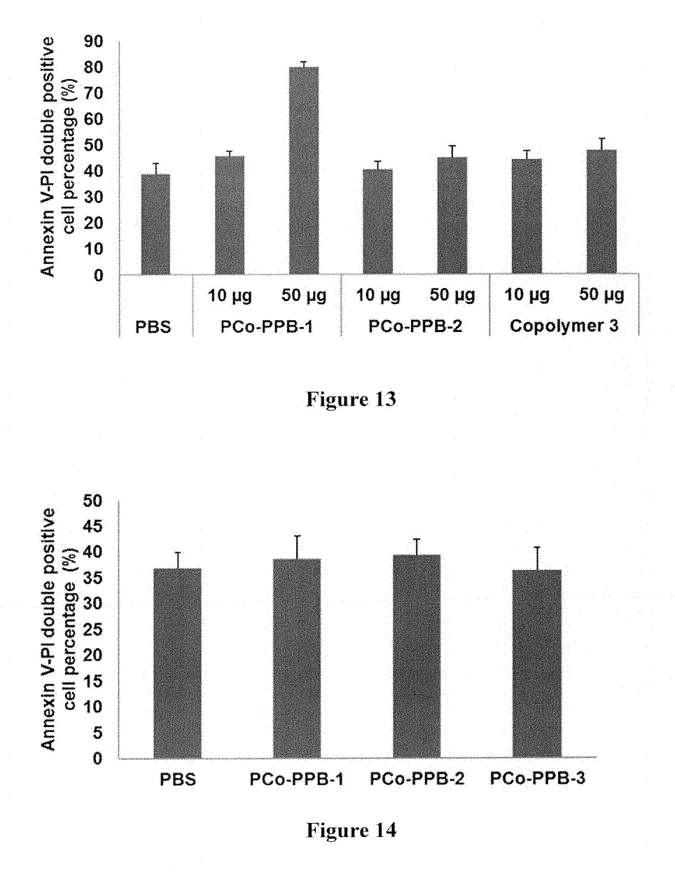

The immune cells were phenotyped for detection of cell subpopulations by targeting their unique markers with a specific antibody followed by detection using flow cytometry, which is a very sensitive technique to quantify large numbers of cells. Splenocytes (1×10⁶) from PBS-treated groups or copolymer-treated groups of mice were washed with PBS (INVITROGEN®) and incubated in the dark for 30 min on ice with 0.5 μg of the following anti-mouse primary monoclonal antibodies (mAb): fluorescein isothiocyanate (FITC)-conjugated anti-CD3, phycoerythrin (PE)-anti-CD8 and allophycocyanin (APC)-anti-CD4 (all from BIOLEGEND®, Calif., USA), or FITC-anti-CD19 (BD PHARMINGEN®, San Diego, Calif., USA). For triple-staining studies, directly-conjugated monoclonal antibodies were simultaneously added to the sample. In the current example, flow cytometry was employed to detect whether the cobaltocenium-boronic acid-containing copolymers influenced the different populations of T and B cell lineages in the splenocytes after intraperitoneal injection of copolymers for 48 h. It was observed that the treatment of mice with any of the cobaltocenium-boronic acid-containing copolymers did not alter the percentages of the immune cells when compared to PBS-treated groups. The percentages of all cell types, including CD3+ T cells, CD4+ T helper/regulatory cells, the CD8+ cytotoxic T cells, as well as the CD19+ B cells from mice injected with the copolymers were similar to those of the PBS-injected mice (FIG. 15). This data strongly suggests that the copolymers did not alter the growth of immune cells.

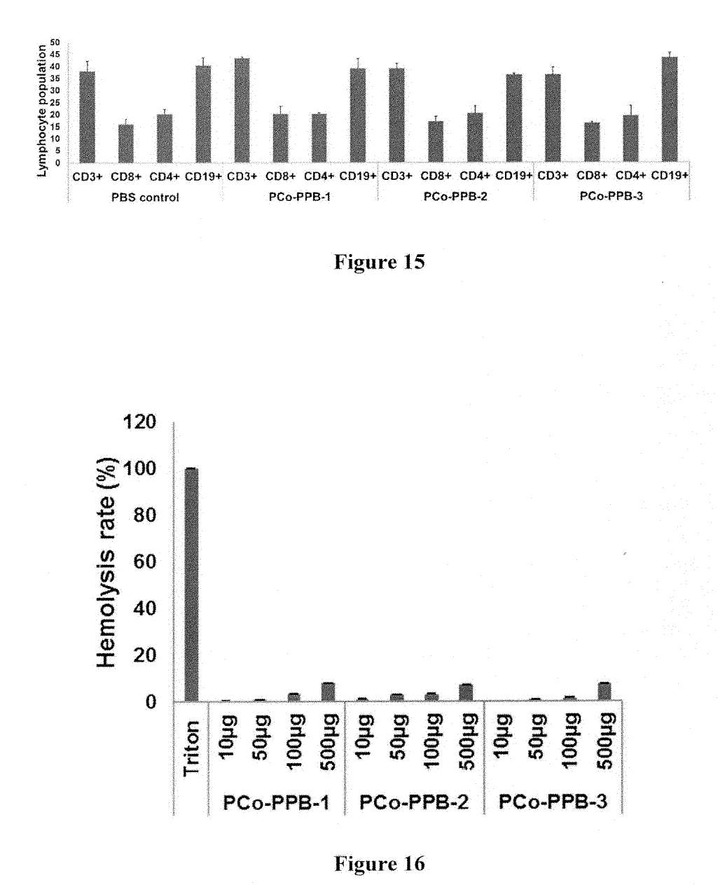

Finally, the toxicity of cobaltocenium-boronic acid-containing copolymers was analyzed on red blood cells (RBCs) by evaluating whether they could lead to hemolysis of red blood cells (RBCs). Blood was collected from mice in heparinized tubes and diluted by mixing 800 μL of blood with 1000 μL PBS. Cobaltocenium-boronic acid-containing copolymer samples were prepared in PBS at concentrations of 10, 50, 100, and 500 μg/mL. Sixty (60) μL of the diluted blood samples were added to 3 mL of each polymer, PBS, and 0.1% Triton-X100 in PBS. Supernatants were then used to measure their optical density (OD) and the hemolysis percentage (%) was calculated. It was found that, even at concentrations of cobaltocenium-boronic acid-containing copolymers as high as 500 μg/mL, all showed lysis of RBCs to be extremely low (<10%) when compared to the negative control group (FIG. 16). Thus, these studies further demonstrated that the cobaltocenium-boronic acid-containing copolymers did not induce cell death by apoptosis, did not alter the phenotypes and the functions of immune cells, and did not show observable toxic effects on RBCs.

In conclusion, the antimicrobial cobaltocenium-boronic acid-containing copolymers exhibited robust, synergistic antibacterial activity through electrostatic absorption onto bacterial membranes/cell walls via the cationic cobaltocenium moiety and the binding of boronic acid to peptidoglycan or lipopolysaccharides on the bacterial surface. Furthermore, these cobaltocenium-boronic acid-containing copolymers possessed excellent biocompatibility. After binding β-lactam antibiotic penicillin-G, the copolymer-antibiotic bioconjugates improved the vitality of antibiotics by protecting the antibiotics from β-lactamase hydrolysis and exhibited excellent antibacterial efficacy against six different strains of Gram-positive and Gram-negative bacteria. This new macromolecular design could open a promising paradigm for improving the vitality of conventional antibiotics against various strains of bacteria while exerting minimal toxicity to mammalian cells.

These and other modifications and variations to the present invention may be practiced by those of ordinary skill in the art, without departing from the spirit and scope of the present invention, which is more particularly set forth in the appended claims. In addition, it should be understood the aspects of the various embodiments may be interchanged both in whole or in part. Furthermore, those of ordinary skill in the art will appreciate that the foregoing description is by way of example only, and is not intended to limit the invention so further described in the appended claims.

What is claimed:

1. A metallocene-boronic acid-containing copolymer comprising at least one metallocene monomer and at least one boronic acid monomer.

2. The metallocene-boronic acid-containing copolymer of claim 1, wherein the metallocene monomer includes a cationic metallocene moiety paired to an anion.

3. The metallocene-boronic acid-containing copolymer of claim 1, wherein the metallocene monomer includes two cyclopentadienyl anions bound to a metal center in oxidation state I.

4. The metallocene-boronic acid-containing copolymer of claim 1, wherein the boronic acid monomer includes a boronic acid group covalently connected to an organic functional group and two hydroxyl groups.

5. The metallocene-boronic acid-containing copolymer of claim 2, wherein the anion includes one or more of a hexafluorophosphate anion, a tetraphenylborate anion, a tetrafluoroborate anion, a trifluoromethanesulfonate anion, F⁻, Cl⁻, Br⁻, I⁻, NO₃⁻, an acetate anion, a sulfate anion, a hydrogen sulfate anion, a perchlorate anion, a bromate anion, a cyanide anion, a thiocyanate anion, a hydroxide anion, a dihydrogen phosphate anion, a formate anion, and mixtures thereof.

6. The metallocene-boronic acid-containing copolymer of claim 2, wherein the anion includes an anionic antibiotic compound.

7. The metallocene-boronic acid-containing copolymer of claim 6, wherein the anionic antibiotic compound includes one or more of a penicillin anion or related compound, a carbapenem anion or related compound, a cephalosporin anion or related compound, and mixtures thereof.

8. The metallocene-boronic acid-containing copolymer of claim 3, wherein the metal center includes one or more of iron, cobalt, rhodium, ruthenium, and mixtures thereof

9. The metallocene-boronic acid-containing copolymer of claim 1, wherein the metallocene-boronic acid-containing copolymer includes a random copolymer.

10. The metallocene-boronic acid-containing copolymer of claim 1, wherein the metallocene-boronic acid-containing copolymer includes a graft copolymer.

11. The metallocene-boronic acid-containing copolymer of claim 1, wherein the metallocene-boronic acid-containing copolymer has a molecular weight of from about 1,000 g/mol to about 1,000,000 g/mol.

12. The metallocene-boronic acid-containing copolymer of claim 1, wherein the at least one metallocene monomer constitutes from about 50 wt. % to about 95 wt. % of the metallocene-boronic acid-containing copolymer.

13. The metallocene-boronic acid-containing copolymer of claim 1, wherein the at least one boronic acid monomer constitutes from about 5 wt. % to about 50 wt. % of the metallocene-boronic acid-containing copolymer.

14. The metallocene-boronic acid-containing copolymer of claim 6, wherein the molar ratio of the anionic antibiotic compound to the at least one metallocene monomer (mols antibiotic/mols metallocene monomer) is from about 0.3 to about 1.0.

15. The metallocene-boronic acid-containing copolymer of claim 6, wherein the anionic antibiotic constitutes from about 10 wt. % to about 50 wt. % of the metallocene-boronic acid-containing copolymer.

16. A method of producing an antibacterial compound comprising polymerizing at least one metallocene monomer with at least one boronic acid monomer.

17. The method of producing an antibacterial compound of claim 16, wherein the at least one metallocene monomer is loaded with an antibiotic.

18. The method of producing an antibacterial compound of claim 17, wherein the at least one metallocene monomer is cationic and the antibiotic is anionic.

19. The method of producing an antibacterial compound of claim 17, wherein the at least one metallocene monomer includes two cyclopentadienyl anions bound to a metal center in oxidation state I.

20. A method of treating bacterial infection in a subject comprising administering the metallocene-boronic acid-containing copolymer of claim 1 to the subject.
